(19) United States
(12) Patent Application Publication
(10) Pub. No.: US 2010/0160873 A1
(43) Pub. Date: Jun. 24, 2010

(54) ANTIMICROBIAL GEL DISPENSER

(76) Inventor: NANCY GOLDRAIN, CARMEL, CA (US)

Correspondence Address: KLEINBERG & LERNER, LLP, 1875 CENTURY PARK EAST, SUITE 1150, LOS ANGELES, CA, 90067, US

(21) Appl. No.: 12/642,134

(22) Filed: Dec. 18, 2009

Related U.S. Application Data
(60) Provisional application No. 61/114,597, filed on Dec. 22, 2008.

(51) Int. Cl. A61M 35/00

(52) U.S. Cl. 604/311; 604/310

(57) ABSTRACT

A tube dispenser adapted to apply antimicrobial gel beneath a user's fingernails includes a rotatable knob coupled to a piston with a threaded rod so that rotation of the knob advances the piston, extruding antimicrobial gel through an applicator tip.

This is a non-provisional application corresponding to provisional application 61/114,579, filed Dec. 22, 2008, the priority of which is claimed.

### BACKGROUND OF THE INVENTION

1. Field of the Invention

The present invention relates in general to a device for personal grooming and, more particularly, to a device for applying an antimicrobial gel to areas underneath the fingernails

2. Description of the Related Art

Ever since soaps were introduced, people have been coming up with new ways to clean and remove dirt and bacteria from the human body. Initially, bar soaps were used to wash skin surfaces and, over time, new methods and devices were introduced to more effectively scrub and clean the body. Liquid soaps were developed to work with sponges and scrubs and other applicators to lather up and more effectively clean different areas of the body. However, as people are not always near water, it became more difficult to take advantage and make use of these various devices and inventions for those who are not always near a source of water.

Recently, antimicrobial gels and waterless sanitizers have been developed to safely remove dirt, germs and bacteria from the human body without the need of water as a source to lather up. They combine the pleasant scent associated with a regular bar soap or liquid soap with the ability to kill germs and bacteria that live on the skin surface. Further, these gels dry without the need of a towel.

The antimicrobial gels and waterless sanitizers are effective in killing 99.9% of the bacteria that live on the surface of the skin. They come in variety of shapes and sizes of bottles for people on the go. However, they are not effective in killing germs that live under the fingernails. It is believed that a significant population of the bacteria on the hand resides under the fingernails. These gels applied to the hand generally do not alone rid the hand of the undernail bacteria population.

Further, appearance of a “French nail” or having assortment of colors painted to the area beneath the fingernails is becoming more popular among the female population. These paintings enhance the difficulty of antimicrobial gels and waterless sanitizers gaining access to the areas beneath the fingernails.

It can be seen, then, that there is a need in the art for an antimicrobial gel applicator that can reach the areas underneath the fingernails. It can also be seen that there is a need in the art for an antimicrobial gel applicator that can remove debris from underneath the fingernails. It can also be seen that there is a need in the art for an antimicrobial gel applicator that can dispense colored antimicrobial gels.

### SUMMARY OF THE INVENTION

To minimize the limitations in the prior art, and to minimize other limitations that will become apparent upon reading and understanding the present specification, the present invention discloses an apparatus for applying an antimicrobial gel to areas underneath the fingernail and also for removing debris underneath the fingernail. An apparatus for dispensing antimicrobial gel includes a body, adapted to hold antimicrobial gel, and an applicator tip, attached to the body, with means to dispense and distribute the antimicrobial gel to the areas underneath the fingernail.

Such a device can further includes a detachable cap that can be secured to the body, whose end is shaped for removing debris from underneath the fingernail.

Accordingly, it is an object of the present invention to provide a device that allows users to apply an antimicrobial gel to the areas underneath the fingernail. It is another object of the present invention to provide an assortment of colors for the antimicrobial gel for fingernail painting. It is still another object of the present invention to provide a device that allows users to safely remove debris from areas underneath the fingernail.

The novel features which are characteristic of the invention, both as to structure and method of operation thereof, together with further objects and advantages thereof, will be understood from the following description, considered in connection with the accompanying drawings, in which the preferred embodiment of the invention is illustrated by way of example. It is to be expressly understood, however, that the drawings are for the purpose of illustration and description only, and they are not intended as a definition of the limits of the invention.

### BRIEF DESCRIPTION OF THE DRAWINGS

Referring now to the drawings in which like reference numbers represent corresponding parts throughout:

FIG. 1 is a perspective view of the apparatus of the present invention.

FIG. 2 is a cut away view of the actuator assembly at the base of the apparatus.

FIG. 3 is an embodiment of, the applicator tip used for the apparatus of the present invention.

FIG. 4 is a side view of the apparatus in an alternate embodiment.

### DETAILED DESCRIPTION OF THE DRAWINGS

In the following description of the preferred embodiment, reference is made to the accompanying drawings that forms a part hereof, and in which is shown by way of illustration a specific embodiment in which the invention may be practiced. It is to be understood that other embodiments may be utilized and structural changes may be made without departing from the scope of the present invention.

### SPECIFICS OF THE INVENTION

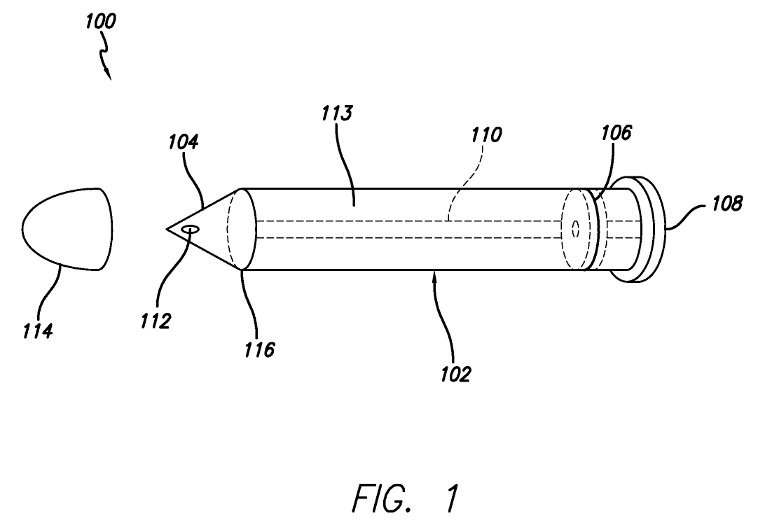

FIG. 1 is a perspective side view of the applicator of the present invention. Applicator 100 includes a hollow body portion 102, applicator tip 104, piston 106, knob 108, a threaded rod 110, and tip cover 114. Although shown as a single unit, and preferably, in final form comprises a single unit, applicator 100 can be manufactured as a combination of the individual elements, if desired.

Applicator 100 is typically cylindrical, and is typically made from plastic. However, the applicator 100 can be made in any shape which can carry a liquid or gel without departing from the scope of the present invention. Further, applicator 100 can be made from metal, glass, or any other non-porous, sustainable material, or combinations of these materials, without departing from the scope of the present invention.

An applicator tip 104, which can be permanent or replaceable, is affixed to the open end of the body portion 102. The tip 104 is provided with an opening 112 through which the antimicrobial gel can be dispensed. The piston 106 has a central opening which is engaged by the threaded rod 110 which engages the piston 106 central opening. The knob 108 is attached to the rod 110 but is exterior to the body portion 102 of the applicator 100. Rotation of the knob 108 causes the piston 106 to advance and retract as it engages the threads of the rod 110.

The tip cover 114 has a shape complementary to the tip 104 and can be used in a manner similar to an “orange stick” to clear debris from under the nail before applying the antimicrobial gel. The cover 114 serves the additional function of covering the opening 112 so that the gel within the dispenser is not exposed to the environment. With the cover 114 in place, there is less likelihood that the gel will dry or evaporate.

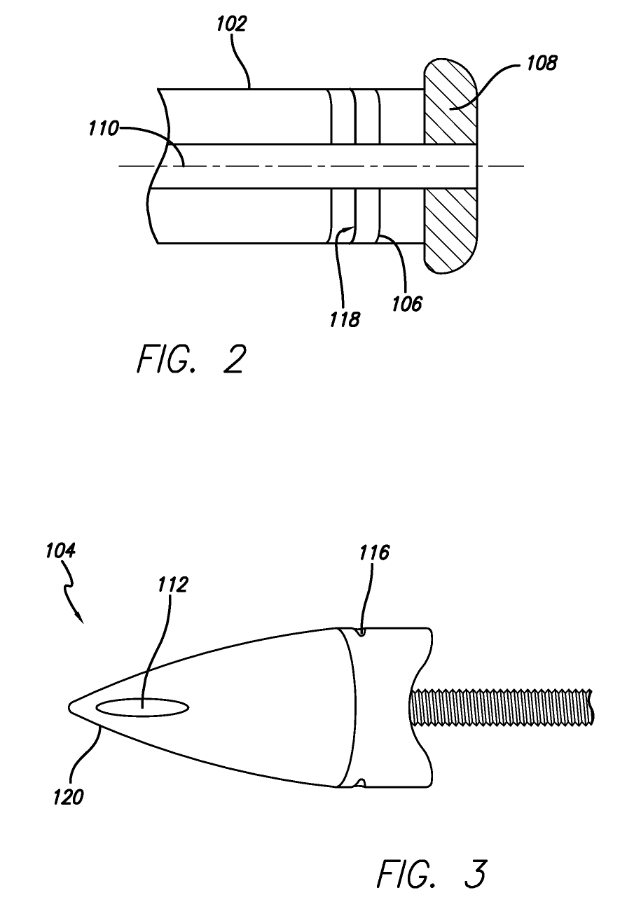

FIG. 2 is a cut away view of the applicator assembly at the base of the apparatus. As shown in FIG. 2, piston 106 can further include a seal ring 118 to prevent antimicrobial gel from leaking around the piston 106. Piston 106 rides within body portion 102 as it responds to the rotation of the rod 110. With the rod 110 and the piston 106 in place within the body portion 102, the body portion 102 can be filled with an antibacterial gel 113 and the tip 104 affixed. Rotation of the knob 108 causes the piston 106 to extrude the gel 113 through the opening 112 of the tip 104 and the tip 104 can apply the gel 113 to the area beneath the fingernail (not shown).

FIG. 3 illustrates an embodiment of the applicator tip 104 used for the applicator 100 of the present invention. As shown in FIG. 3, applicator tip 104 further includes a ridge 116, and tapered portion 120. The Applicator tip 104 typically is held onto the body 102 as a snap fit over a ridge 116 in the body portion 102. However, applicator tip 104 can be held onto the body portion 102 by any other means that substantially seals applicator tip 104 and body portion 102 together, without departing from the scope of the present invention. Upon rotation of the rod 110, the applicator tip 104 will dispense antimicrobial gel 113 from the interior of the body portion 102 as the piston 106 responds to the rotation of the rod 110. The gel 113 flows through interior passages of the tip 104 and out of the opening 112.

Applicator tip 104 typically has a tapered shape to more easily reach the area beneath the fingernail, however, there may be situations where a shape other than tapered for surface of the applicator tip 104 may be desired, and the use of such shapes would not depart from the scope of the present invention. Applicator tip 104 can also be manufactured as part of the body portion 102.

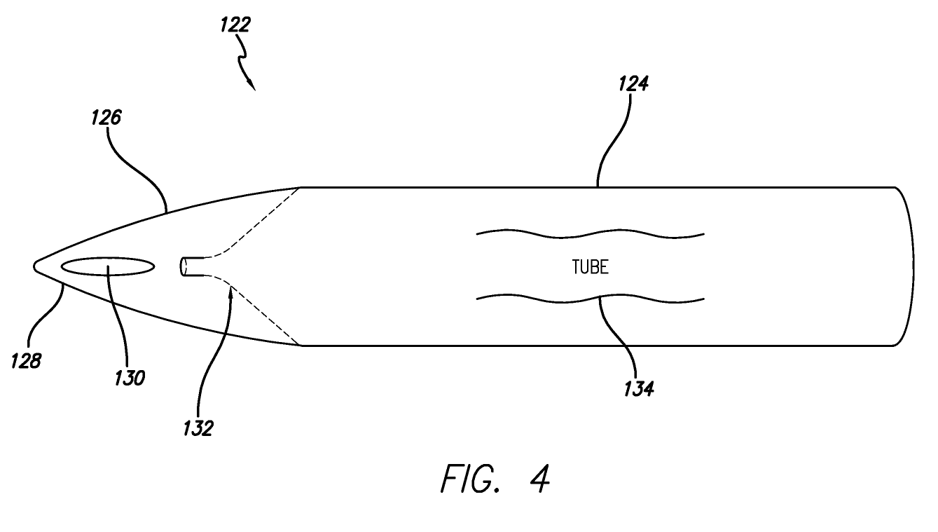

In FIG. 4, a side view of the apparatus is shown in an alternate embodiment. Applicator 122 is shown in FIG. 4, with body portion 124, applicator tip 126, and opening 130 as indicated.

Body portion 124 is typically a flexible tube, and is typically made from a nonporous material. However, body portion 124 can be a bottle, a vessel, a closed cup, or other liquid or gel carrying device without departing from the scope of the present invention. Further, body portion 124 can be made from plastic, or other materials or combinations of these materials without departing from the scope of the present invention. A flexible, or deformable body will permit dispensing of the gel by application of manual pressure to the body portion. Rigid embodiments will require additional means to transfer gel from the body portion interior to an applicator tip 126.

Applicator tip 126 includes a tapered portion 128 that is typically flat such that it can more easily reach the area beneath the fingernail for applying antimicrobial gel and removing debris. Applicator tip 126 further includes opening 130, and internal flow passages or conduits 132 that allow antimicrobial gel 134 to flow through the tip opening 130. Conduit 132 will flow antimicrobial gel 134 from the interior of body portion 124 upon squeezing the body portion 124, causing the gel 134 to extrude through the opening 130 of the tip 126 and the tip 126 can apply the gel 134 to the area beneath the fingernail.

### CONCLUSION

In Summary, the present invention provides-an applicator for containing antimicrobial gel and means for both cleaning and applying antimicrobial gel to the area underneath the fingernail. An apparatus in accordance with the present invention comprises a body, applicator tip, and means for dispensing antimicrobial gel.

The foregoing description of the preferred embodiments of the invention has been presented for the purpose of illustration and description. It is not intended to be exhaustive or to limit the invention to the precise forms disclosed. Many modifications and variations are possible in light of the above teaching. It is intended that the scope of the invention not be limited by this detailed description, but by the claims and the equivalents to the claims that are appended hereto.

What is claimed is:

1. An apparatus for dispensing antimicrobial gel comprising:
a body, adapted to hold antimicrobial gel; and
an applicator tip shaped to fit beneath a fingernail tip, attached to said body, said body having means to dispense antimicrobial gel.

2. The apparatus of claim 1, wherein said means to dispense antimicrobial gel include a knob attached to the end of the body, coupled to piston means within said body for extruding antimicrobial gel through said applicator tip.

3. The apparatus of claim 1, wherein said means to dispense antimicrobial gel include a rod extending into said body, and knob means, exterior to said body and attached to said rod, whereby rotation of said knob means controls extrusion of antimicrobial gel.

4. The apparatus of claim 3, wherein said means to dispense antimicrobial gel include a piston inside said body and attached to said rod, said rod being threaded for engaging said piston and wherein said piston advances and retracts within said body with rotation of said knob.
